(19) United States
(12) Patent Application Publication
(10) Pub. No.: US 2021/0069012 A1
(43) Pub. Date: Mar. 11, 2021

(54) SURFACE COOLING AND HEATING DEVICES AND METHODS OF USE

(71) Applicant: IMPACT TECHNOLOGIES, LLC, Cleverdale, NY (US)

(72) Inventor: Jason E. Kirshon, Cleverdale, NY (US)

(73) Assignee: IMPACT TECHNOLOGIES, LLC, Cleverdale, NY (US)

(21) Appl. No.: 17/015,738

(22) Filed: Sep. 9, 2020

Related U.S. Application Data
(60) Provisional application No. 62/898,365, filed on Sep. 10, 2019.

(51) Int. Cl. A61F 7/03; A61F 7/10

(52) U.S. Cl. CPC A61F 7/03; A61F 7/106

(57) ABSTRACT

Surface treatment devices having a flexible, fluid impermeable enclosure made of a synthetic amorphous silica; and a polydimethlysiloxane fluid contained in the enclosure. The synthetic amorphous silica may be a liquid silicone rubber, for example, s CHT True Skin liquid silicone rubber. The polydimethlysiloxane fluid may be a CHT QM Diluent. The treatment device may be used for heating or cooling a surface, for example, the surface of the human body. The liquid silicone rubber may be a CHT True Skin 10 liquid silicone rubber, and the CHT QM Diluent may be a low viscosity CHT QM Diluent, for example, a CHT QM Diluent having a viscosity of at least 50 centipoise (cps), or at least 1,000 cps. Methods for treating a surface and methods for fabricating a surface treating device are also provided.

### CROSS-REFERENCE TO RELATED APPLICATION

This application claims priority from pending U.S. Provisional Patent Application 62/898,365 filed on Sep. 10, 2019, the disclosure of which is included by reference herein in its entirety.

### BACKGROUND OF THE INVENTION

### Technical Field

Aspects of the present invention relate generally to surface treatment devices, such as, heating devices and/or cooling devices. More particularly, aspects of the invention, in their several embodiments, provide surface treatment devices fabricated from liquid silicone rubber and containing and a polydimethlysiloxane fluid that provide enhanced heating and/or cooling, for example, to treating skin surfaces and the underlying tissue, for instance, for treatment of injuries.

### Description of Related Art

Numerous human activities, such as, recreation and sports, public protection, and armed service expose the human body, to impact and injury. The prior art includes many forms of “heating pads” and/or “ice packs” that can effectively aid in the treatment of such bodily injuries. However, there continues to be a need in the art to provide more effective devices and methods for promoting the alleviation of pain and to enhance healing.

### SUMMARY OF THE INVENTION

Embodiments of the present invention, in their many aspects, provide improved devices and methods for treating tissue damage or injuries to, among other things, enhance healing and alleviate discomfort.

Though aspects of the present invention may be used for treating bodily ailments of humans or animals, for example, providing medical treatment or medical devices, aspects of the invention may also be used to treat any surface for which thermal treatment, that is, heating and/or cooling, is desirable. Possible non-medical surface treatment includes heat treatment or cooling during fabrication processes, among other non-medical applications.

One embodiment of the invention is a surface treatment device comprising or including: an enclosure comprising a CHT True Skin liquid silicone rubber; and a CHT QM Diluent contained in the enclosure. In one aspect, the device may be a heating device and/or a cooling device.

In one aspect, the CHT True Skin liquid silicone rubber may be a CHT True Skin liquid silicone rubber, for example, a CHT True Skin 10 liquid silicone rubber produced by combining at least two liquids.

In one aspect the CHT QM Diluent may be a CHT QM Diluent, for example, a CHT QM Diluent having a viscosity of at least 50 cps, or a CHT QM Diluent having a viscosity of at least 1000 cps, or a CHT QM Diluent having a viscosity of at least 5,000 cps.

Another embodiment of the invention is a surface treatment device comprising or including a flexible, fluid impermeable enclosure comprising synthetic amorphous silica; and a polydimethlysiloxane fluid contained in the enclosure. In one aspect, the device may be a cooling device and/or a heating device.

In one aspect, the synthetic amorphous silica may be a CHT True Skin liquid silicone rubber, for example, a CHT True Skin 10 liquid silicone rubber.

In one aspect, the polydimethlysiloxane fluid may be a CHT QM Diluent, for example, a CHT QM Diluent having a viscosity of at least 50 centipoise [cps], or a CHT QM Diluent having a viscosity of at least 1000 cps, or a CHT QM Diluent having a viscosity of at least 5,000 cps.

Another embodiment of the invention is a method for treating a surface comprising: heating or cooling a flexible, fluid impermeable enclosure comprising a synthetic amorphous silica and containing a polydimethlysiloxane fluid; placing the heated or cooled flexible enclosure upon the surface; and conducting thermal energy from the enclosure to the surface or conducting thermal energy from the surface to the enclosure. In one aspect, heating may be practiced by heating in a heating device and cooling may be practiced by cooling in a cooling device.

Another embodiment of the invention is a surface treatment device comprising or including: a flexible, fluid impermeable enclosure comprising a plurality of interconnected components comprising a synthetic amorphous silica; and a polydimethlysiloxane fluid contained in the enclosure. The device may be a heating device and/or a cooling device.

In one aspect, the plurality of interconnected components may comprise a plurality of interconnected cylindrical enclosures, for example, a plurality of interconnected circular cylindrical enclosures or a plurality of interconnected polygonal cylindrical enclosures. In one aspect, the plurality of interconnected cylindrical enclosures may be in fluid communication. In another aspect, the plurality of interconnected cylindrical enclosures may include internal supports, such as, posts or pins. In one aspect, the internal supports may be hollow internal supports.

In one aspect, the synthetic amorphous silica of the plurality of interconnected components may be a CHT True Skin liquid silicone rubber. In one aspect, the polydimethlysiloxane fluid within the plurality of interconnected components may be a CHT QM Diluent.

A still further aspect of the invention is a method for fabricating a surface treating device, the method comprising or including: providing a first mold adapted to receive a first curable fluid, the first mold having mold cavity and a lower surface and a plurality of projections from the lower surface; introducing a first curable fluid to the first mold to at least partially cover the lower surface with the first curable fluid; allowing the first curable fluid to cure on the lower surface to produce an at least partially cured first device portion; mating the first device portion with a second device portion to form a fluid-impermeable enclosure; and introducing a fluid to the fluid-impermeable enclosure to provide the surface treatment device.

In one aspect, mating the first device portion with the second device portion comprises sealing peripheral edges of the first device portion with peripheral edges of the second device portion.

In one aspect, the plurality of projections from the lower surface comprise a plurality of cylindrical projections.

In one aspect, the first curable fluid comprises a liquid silicone rubber, for example, a CHT True Skin liquid silicone rubber. In another aspect, introducing the fluid, for example, a CHT QM Diluent, to the fluid-impermeable enclosure is practiced by injecting the fluid into the fluid-permeable enclosure.

These and other aspects, features, and advantages of this invention will become apparent from the following detailed description of the various aspects of the invention taken in conjunction with the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other objects, features, and advantages of the invention will be readily understood from the following detailed description of aspects of the invention taken in conjunction with the accompanying drawings in which:

FIG. 1 is a schematic perspective view of a device, for example, a heat/cold pack, according to one aspect of the invention.

FIG. 2 is a top plan view of the device shown in FIG. 1, the bottom view being a mirror image thereof.

FIG. 3 is a side elevation view of the device shown in FIG. 1.

FIG. 4 is a schematic perspective view of another device according to one aspect of the invention.

FIG. 5 is a top plan view of the device shown in FIG. 4, the bottom view being a mirror image thereof.

FIG. 6 is a side elevation view of the device shown in FIG. 4.

FIG. 7 is a perspective view of the aspect of the invention shown in FIGS. 4 through 6 as typically used upon a human knee.

FIG. 8 is a perspective view of another device according to an aspect of the invention.

FIG. 9 is a top plan view of the device shown in FIG. 8.

FIG. 10 is a bottom plan view of the device shown in FIG. 8.

FIG. 11 is a perspective view of a detail of the device shown in FIG. 8.

FIGS. 12 through 17 are schematic plan views of alternate shapes of surface treatment devices according to aspects of the invention.

### DETAILED DESCRIPTION OF THE INVENTION

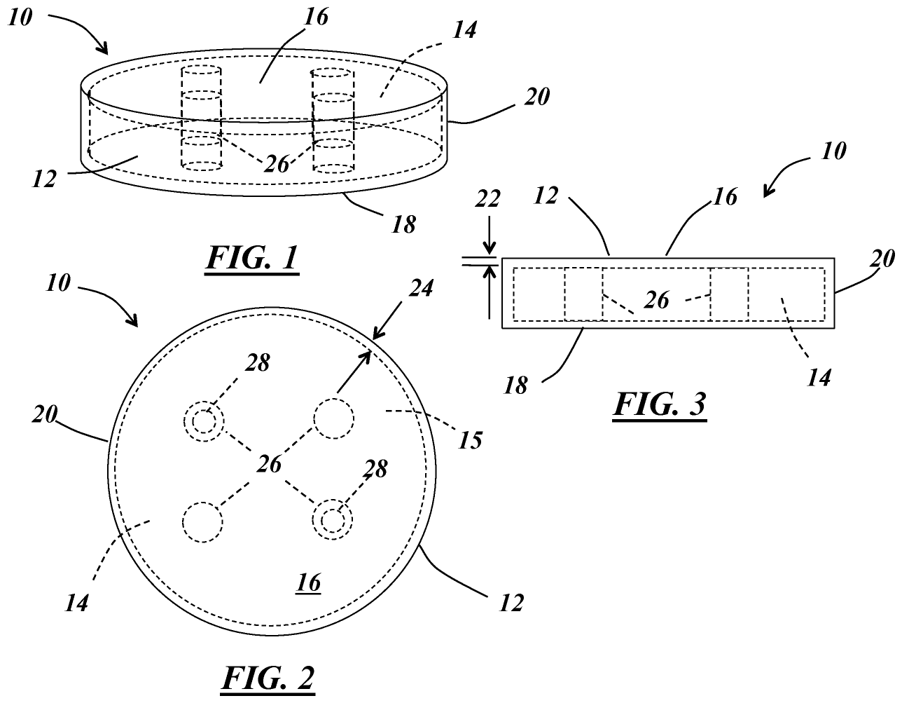

FIG. 1 is a schematic perspective view of a device 10, for example, a dermatological device, for cooling and/or heating an object or a surface, for example, for cooling or heating the skin and/or underlying body structures lying beneath the skin, for example, of a human or an animal. In one aspect, device 10 (and any device disclosed herein) may be used as an “ice pack” for treating a bodily surface and/or the underlying tissue with cold, for example, a twisted ankle or a forehead of a person with a headache. In another aspect, device 10 (and any device disclosed herein) may be used as a “heat pack” for treating a bodily surface and/or the underlying tissue with heat, for example, a strained knee or pulled back muscle. In another aspect, device 10 (and any device disclosed herein) may be used for comfort, cushioning, and/or padding, for example, as a replacement or substitute for “comfort foam,” for example, with little or no cooling or heating. For instance, in one aspect, device 10 (and any device disclosed herein) may be used with prosthetics or orthotics to reduce or alleviate any discomfort related to the prosthetics or orthotics. FIG. 2 is a top plan view of the device 10 shown in FIG. 1, the bottom view being a mirror image thereof, and FIG. 3 is a side elevation view of the device 10 shown in FIG. 1.

Though in the following discussion device 10 and related devices disclosed herein may be disclosed and described with respect to the treatment of bodily surfaces, it is envisioned that aspects of the invention may be used for treating any surface, bodily or otherwise, for which heating and/or cooling is desired or beneficial, for example, the heating and/cooling of food or beverages, and/or the heating and/or cooling of any surface during an industrial and/or fabrication process, and/or the heating and/or cooling of any other surface that would benefit from heating and/or cooling.

As shown in FIGS. 1 through 3, device 10 comprises an enclosure 12 having a fluid 14 encased within enclosure 12. According to aspects of the invention, device 10 (and any device disclosed herein) may be cooled or heated, for example, prior to being applied to the surface being treated, and the retained temperature of the heating or cooling of device 10 may be used to treat a surface, for example, the surface of skin, upon which device 10 is placed. Though in one aspect device 10 may be placed directly upon the surface of the skin, for example, of human skin, the benefits of aspects of the invention may be effective in cooling or heating skin or other surface through an intermediate barrier, for example, when encased in a package or wrapped in a fabric.

In one aspect, enclosure 12 of device 10 may comprise a liquid silicone rubber (LSR), for example, a cured LSR, such as, a “medical grade” LSR, as known in the art. In one aspect, enclosure 12 of device 10 may comprise a liquid silicone rubber marketed by CHT USA (formerly Quantum Silicones (QSi)) under the trademark True Skin® or its equivalent; however, enclosure 12 may comprises any liquid silicone rubber (LSR).

In one aspect, the liquid 14 encased in enclosure 12 may comprise a polydimethlysiloxane fluid (also know as a “silicone fluid), for example, a polydimethlysiloxane fluid marketed by CHT USA as a QM Diluent or its equivalent; however, liquid 14 may comprise any polydimethlysiloxane fluid. As known in the art, polydimethlysiloxane comprises a group of polymeric organosilicon compounds that are commonly referred to as “silicones.” Polydimethlysiloxane may be abbreviated “PDMS’ and has the chemical formula [(CH₃)₂SiO]ₙ. However, in other aspects, liquid 14 may be any fluid, including water.

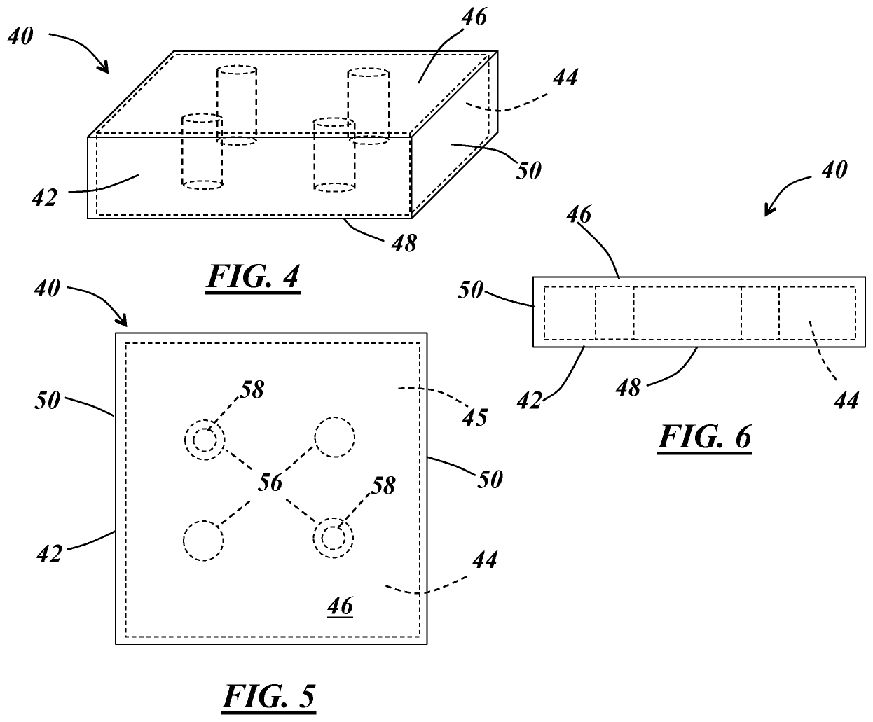

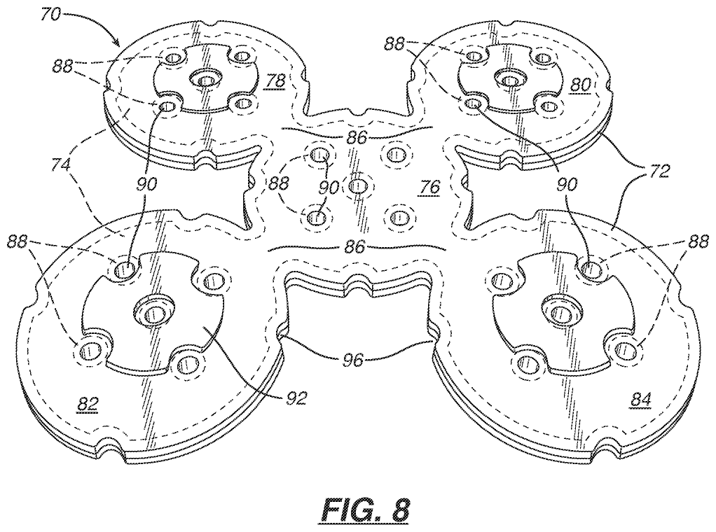

According to aspects of the invention, enclosure 12 of device 10 may take any conventional size and shape, for example, depending upon the size and/or shape of the surface being treated, for example, a human shoulder or human knee. As shown in FIGS. 1 through 3, in one aspect, device 10 may be cylindrical in shape, for example, right circular cylindrical in shape. As shown in FIGS. 4 through 6, in one aspect, device 10 may be polygonal cylindrical in shape, for example, rectangular cylindrical in shape or square cylindrical in shape, pentagonal cylindrical in shape, or hexagonal cylindrical in shape, among other cylindrical shapes. Other shapes for enclosure 12 are also envisioned, for example, as shown in FIGS. 8 through 17.

As shown in FIGS. 1 through 3, enclosure 12 may comprise any enclosure having an internal cavity 15 containing liquid 14. According to aspects of the invention, enclosure 12 may be liquid impermeable, for example, at least impermeable to the passage of liquid 14. As shown in FIGS. 1 through 3, enclosure 12 may typically have opposing end walls 16 and 18 (for example, a top and a bottom) and at least one sidewall 20. End walls 16 and 18 (and any end walls disclosed herein) have a thickness 22, for example, a common thickness (though the thickness of the end walls 16 and 18 may vary) and sidewall 20 (and any sidewall disclosed herein) has a thickness 24. The thicknesses 22 and 24 may be equal or vary, and may typically range from about 0.05 inches to about 0.25 inches, for example, between about 0.10 inches to about 0.12 inches.

In one aspect of the invention, the internal cavity 15 of device 10 may devoid of any structure or obstruction. However, in another aspect of the invention, the internal cavity 15 may comprise one or more supports or structures positioned and adapted to retain the shape of device 10, for example, preventing deflection of end walls 16 or 18 and or the deflection of sidewall 20. As shown in phantom in FIGS. 1 through 3, enclosure 12 of device 10 may include one or more structures, for example, pins, rods, or supports 26 adapted to engage opposing end walls 16 and 18 and, for example, prevent portions of end walls 16 or 18 from deflecting, for example, under the force of gravity or the force of the pressure of fluid 14. As shown in FIGS. 1 through 3, in one aspect, supports 26 may be cylindrical in shape, for example, circular cylindrical, though any cylindrical shape may be used. In one aspect, supports 26 may be hollow, as indicated by internal cavities 28, two of which are shown representatively in FIG. 2; however, one or more of supports 26 may be hollow in one aspect. In one aspect, hollow supports 26 may penetrate end walls 16 and 18 or be in fluid communication with through holes in end walls 16 and 18, for example, where an open passage is provided though device 10 via hollow supports 26. Supports 26 may be mounted by an adhesive in cavity 15 of enclosure 14 or molded into end walls 16 and 18 of enclosure 14.

FIG. 4 is a schematic perspective view of another device 40 according to one aspect of the invention. FIG. 5 is a top plan view of device 40 shown in FIG. 4, the bottom view being a mirror image thereof, and FIG. 6 is a side elevation view of device 40 shown in FIG. 4. As shown in FIGS. 4 through 6, in this aspect, device 40 may be polygonal cylindrical in shape, for example, rectangular cylindrical in shape or square cylindrical in shape, pentagonal cylindrical in shape, or hexagonal cylindrical in shape, among other cylindrical shapes. Other shapes for enclosure 12 are also envisioned, for example, as shown in FIGS. 8 through 17.

Though the shape of device 40 may be different from the shape of device 10, device 40 may have all the features and attributes of device 10. For example, device 40 comprises an enclosure 42 having a fluid 44 encased within enclosure 42. In one aspect, in a fashion similar to device 10, enclosure 42 of device 40 may comprise a cured liquid silicone rubber, for example, a liquid silicone rubber marketed by CHT USA under the trademark True Skin® or its equivalent; however, enclosure 42 may comprises any liquid silicone rubber (LSR). In one aspect the liquid 44 encased in enclosure 42 may comprise a polydimethlysiloxane fluid (also know as a “silicone fluid), for example, a polydimethlysiloxane fluid marketed by CHT USA as a QM Diluent or its equivalent; however, fluid 44 may comprises any polydimethlysiloxane fluid.

According to aspects of the invention, enclosure 42 of device 40 may take any conventional size and shape, for example, depending upon the size and/or shape of the surface being treated, for example, a human shoulder or a human knee.

As shown in FIGS. 4 through 6, enclosure 42 may comprise any enclosure having an internal cavity 45 containing liquid 44. According to aspects of the invention, enclosure 42 may be liquid impermeable, for example, at least impermeable to the passage of liquid 44. As shown in FIGS. 4 through 6, enclosure 42 may typically have opposing end walls 46 and 48 (for example, a top and a bottom) and at least one sidewall 50.

In one aspect of the invention, the internal cavity 45 of device 40 may devoid of any structure or obstruction. However, in another aspect of the invention, the internal cavity 45 may comprise one or more supports or structures positioned and adapted to retain the shape of device 40, for example, preventing deflection of end walls 46 or 48 and/or the deflection of sidewalls 50. As shown in phantom in FIGS. 4 through 6, enclosure 42 of device 40 may include one or more structures, for example, pins, rods, or supports 56 adapted to engage opposing end walls 46 and 48 and, for example, prevent portions of end walls 46 or 48 from deflecting, for example, under the force of gravity or the force of pressure from fluid 44. As shown in FIGS. 4 through 6, in one aspect, supports 56 may be cylindrical in shape, for example, circular cylindrical, though any cylindrical shape may be used. In one aspect, supports 56 may be hollow, as indicated by internal cavities 58, two of which are shown representatively in FIG. 5, however, one or more of supports 56 may be hollow in one aspect. In one aspect, hollow supports 56 may penetrate end walls 46 and 48 or be in fluid communication with through holes in end walls 46 and 48, for example, where an open passage is provided though device 40 via hollow supports 56. Supports 56 may be mounted by an adhesive in cavity 45 of enclosure 42 or molded into end walls 46 and 48 of enclosure 42.

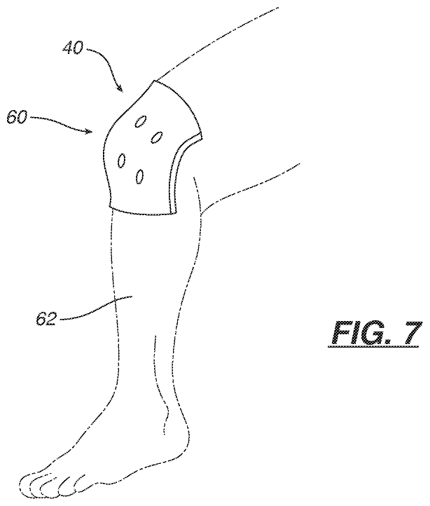

FIG. 7 is a perspective view the device 40 shown in FIGS. 4 through 6 as typically used upon a knee 60 of a human leg 62. As shown in FIG. 7, device 10 or device 40 (or any device disclosed herein), after heating or cooling, may be applied to any area of the body, for example, an injured or a stressed area of the body, to provide at least some comfort and/or enhanced healing. Though in the aspect shown in FIG. 7, the device 40 is shown applied to the knee 60, aspects of the invention may be applied to any bodily surface, including a knee, a foot, an ankle, a leg, an arm, an elbow, a wrist, a back, a shoulder, the buttocks, a chest, an abdomen, or a head, among other areas, and provide at least some beneficial treatment.

According to aspects of the invention, device 10 or device 40 (or any device disclosed herein) may first be heated or cooled prior to application to, for example, to a bodily surface. For example, in one aspect, device 10 or device 40 may be refrigerated in a chilled space, in an icebox, or in ice water, or other chilled fluid to reduce the temperature of device 10. In one aspect, device 10 or device 40 may also be cooled via chemical reaction, for example, by selectively combining reactants within device 10 or device 40 to reduce the temperature of device 10 or device 40. For example, device 10 or device 40 may include one or more reactants contained in a breakable vessel within device 10 or device 40 wherein, when the breakable vessel is broken (for example, by impact or bending), the reactants react and cool at least a portion of device 10 or device 40, for example, via an endothermic reaction. In one aspect, device 10 or 40 may be chilled where the surface of device 10 or device 40 has a temperature of at most 50 degrees F., or at least 30 degrees F., or at least 10 degrees F.

In one aspect of the invention, when treating a surface with colder temperatures, for example, a colder temperature typically not tolerated by human or animal contact, device 10 or device 40 may be cooled to even lower temperatures, for example, lower than 10 degrees F. or lower than zero degrees F., and, due to the low freezing point of the fluid within device 10 or device 40, be used to treat surfaces, for example, during a fabrication process, that can be treated with temperatures below temperatures that can be tolerated by human or animal contact.

After cooling, device 10 or 40 may be contacted with the surface of the body being treated to cool the surface of the skin and/or to cool any underlying tissue. According to one aspect of the invention, device 10 or device 40 may be placed in direct contact with the skin, for example, unlike prior art devices, without the presence of any intervening barrier. In other aspects, an intervening barrier, for example, a container or a fabric, may be placed between device 10 or device 40 and the surface being treated. In another aspect, device 10 or device 40 may be placed in a container or package prior to being applied to the surface of the body being treated.

According to aspects of the invention, device 10 or device 40 may provide an effective means of treating the skin and/or underlying tissue with cooling, for example, to reduce inflammation, cool burns to the skin, or otherwise beneficially treat the skin and/or underlying tissue with reduced temperature.

According to another aspects of the invention, device 10 or device 40 (or any device disclosed herein) may first be heated, for example, in an oven, for instance, a conventional or microwave oven, or by any heat source, to increase the temperature of device 10 or device 40. In one aspect, device 10 or device 40 may also be heated via chemical reaction, for example, by selectively combining reactants within device 10 or device 40 to increase the temperature of device 10 or device 40. For example, device 10 or device 40 may include one or more reactants contained in a breakable vessel within device 10 or device 40 wherein, when the breakable vessel is broken (for example, by impact or bending), the reactants react and heat at least a portion of device 10 or device 40, for example, via an exothermic reaction.

In one aspect, device 10 or 40 may be heated where the surface of device 10 or device 40 is heated to a temperature of at least 50 degrees F., or at least 70 degrees F., or at least 90 degrees F. In one aspect, of the invention, the surface of device 10 or device 40 may be heated to at least 120 degrees F. or 150 degrees F. After heating, device 10 or 40 may be contacted with the surface of the body being treated to heat or warm the surface of the skin and/or to heat or warm any underlying tissue.

In one aspect of the invention, when treating a surface with hotter temperatures, for example, temperatures typically beyond what can be tolerated by human or animal contact, device 10 or device 40 may be used to heat a surface to even higher temperatures, for example, higher than 150 degrees F. or higher than 200 degrees F., and, due to the high boiling point of the fluid within device 10 or device 40, then be used to treat surfaces, for example, during a fabrication process, that can be treated with temperatures above temperatures that can be tolerated by human or animal contact.

According to one aspect of the invention, device 10 or device 40 may be placed in direct contact with the skin to heat or warm the skin, for example, unlike prior art devices, without the presence of any intervening barrier. In other aspects, an intervening barrier, for example, a container or fabric, may be placed between device 10 or device 40 and the surface being treated. In another aspect, device 10 or device 40 may be placed in container or package prior to being applied to the surface of the body being treated.

According to aspects of the invention, device 10 or device 40 may provide an effective means of heating the skin and/or underlying tissue with heat or warmth, for example, to enhance healing of injured tissue, warm an exposed skin surface, or otherwise beneficially treat the skin and/or underlying tissue with increased temperature.

In another aspect, device 10 or device 40 may be used for comfort, cushioning, and/or padding, for example, as a replacement or substitute for “comfort foam,” for example, with little or no cooling or heating.

As described above, aspects of the present invention may be provided in many different sizes and shapes while effectively providing the desired cooling, heating, and/or comfort. For example, the sizes and shapes of the cylindrical enclosure 12 shown in FIGS. 1 through 3 and cylindrical enclosure 42 shown in FIGS. 4 through 6 may vary broadly depending upon the application or use of aspects of the invention, for instance, the size and/or location of the portion of the body being treated and/or the desired length of time of heating and/or cooling treatment.

In one aspect, the thickness, or shortness dimension of enclosure 12 or of enclosure 42 may vary from 0.125 inches to 12 inches, but is typically between 0.25 inches and 3 inches, for example, about 0.375 inches thick. In one aspect, the outside width or outside diameter, or largest dimension, of enclosure 12 or of enclosure 42 may vary from 1 inch to 36 inches, but is typically between 3 inches and 12 inches, for example, about 6 inches wide.

In addition, it is envisioned that aspects of the invention need not be limited to simple cylindrical shapes as shown in FIGS. 1 through 6, but that a broad range of irregular, multi-lobed, or multi-component shapes may be used for enclosure 12 or for enclosure 42. FIGS. 8 through 11 illustrate one of the many shapes of enclosures that may be used to provide aspects of the present invention and the benefits of those aspects.

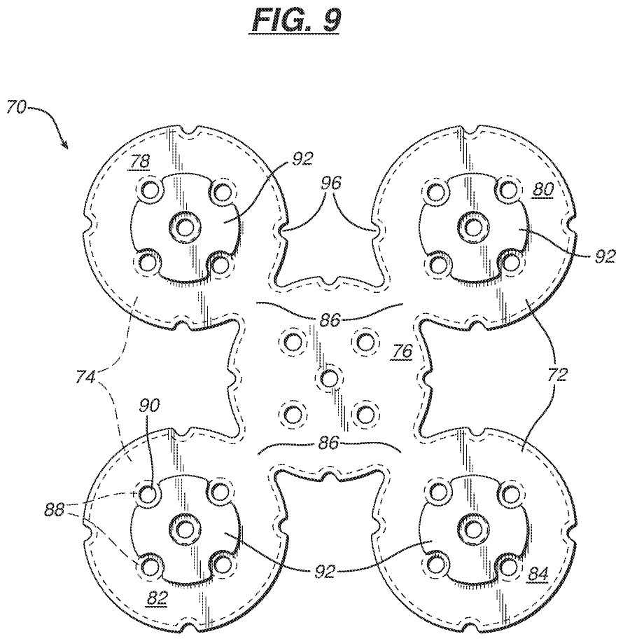

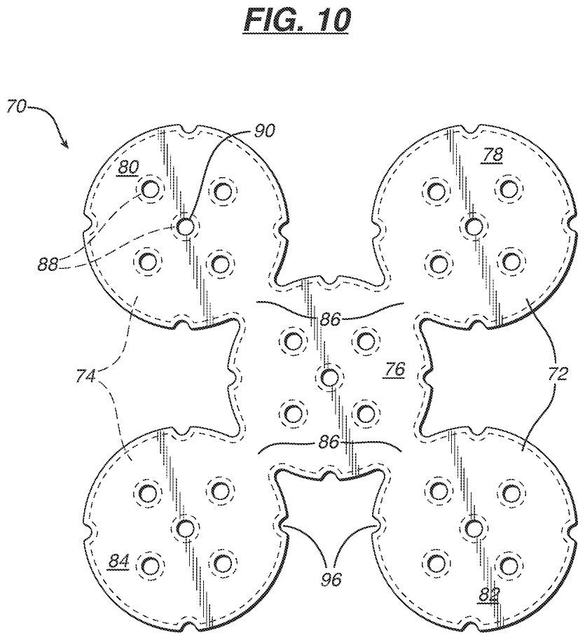

FIG. 8 is a perspective view of surface treatment device 70 according to another embodiment of the invention. FIG. 9 is a top view of the treatment device 70 shown in FIG. 8 and FIG. 10 is a bottom plan view of the treatment device 70 shown in FIG. 8. FIG. 11 is a perspective view of a detail of the treatment device 70 shown in FIG. 8.

As shown in FIGS. 8 through 11, similar to devices 10 and 40 disclosed herein, device 70 comprises an enclosure 72 having a fluid 74 encased within enclosure 72. According to aspects of the invention, device 70 (and any device disclosed herein) may be cooled or heated, and the retained temperature of the heating or cooling of device 70 may be used to treat a surface, for example, the surface of skin, upon which device 70 is placed. Though in one aspect device 70 may be placed directly upon the surface of the skin, for example, of human skin, the benefits of aspects of the invention may be effective in cooling, heating, and/or comforting skin through an intermediate barrier, for example, when encased in a package or wrapped in a fabric.

In one aspect, enclosure 72 of device 70 may comprise a liquid silicone rubber, for example, a liquid silicone rubber marketed under the trademark True Skin®, or its equivalent; however, enclosure 72 may comprises any cured liquid silicone rubber (LSR). In one aspect the liquid 74 encased in enclosure 12 may comprise a polydimethlysiloxane fluid, for example, a polydimethlysiloxane fluid marketed as a QM Diluent, or its equivalent; however, liquid 74 may comprises any polydimethlysiloxane fluid.

According to aspects of the invention, as shown in FIGS. 8 through 11, device 70 may comprise a multi-component cylindrical shape, for example, a plurality of interconnected right circular cylindrical components 76, 78, 80, 82, and 84 having fluid 74. As shown in FIGS. 8 through 11, in one aspect, device 70 may comprise five circular cylindrical components, for example, five interconnected devices 10 or devices 40 as shown in FIGS. 1 through 6. However, it is envisioned that aspects of the invention may have 2 or more interconnected components 76, 78, 80, 82, and 84. In one aspect, as shown in FIGS. 8 through 11, at least some of components 76, 78, 80, 82, and 84, but typically all of components 76, 78, 80, 82, and 84, may be in fluid communication with at least one of the other components 76, 78, 80, 82, and 84. However, in other aspects, at least one of the 2 or more interconnected components 76, 78, 80, 82, and 84, for example, all of the interconnected components 76, 78, 80, 82, and 84, may not be in fluid communication but may be in fluid isolation from each other. For example, in one aspect, one or more of the components 76, 78, 80, 82, and 84 may contain a different fluid, for example, a different QM Diluent or an entirely different fluid.

As shown in FIGS. 8 through 11, in one aspect, the components 76, 78, 80, 82, and 84 of device 70 may be connected to at least one of the other components 76, 78, 80, 82, and 84 by an interface, transition, extension, or projection 86 between components 76, 78, 80, 82, and 84. According to aspects of the invention, interface, transition, extension, or projection 86 may be hollow, for example, allowing fluid communication between components; or solid, for example, preventing fluid communication between components.

In a fashion similar to devices 10 and 40 disclosed herein, the internal cavities of components 76, 78, 80, 82, and 84 of device 70 may be devoid of any structures or obstructions. However, in another aspect of the invention, the internal cavities of components 76, 78, 80, 82, and 84 may comprise one or more supports or structures positioned and adapted to retain the shape of the components 76, 78, 80, 82, and 84 of device 70, for example, preventing deflection of end walls and/or the deflection of sidewalls. As shown in FIGS. 7 through 11, components 76, 78, 80, 82, and 84 of device 70 may include one or more structures, for example, pins, rods, or supports 88 adapted to engage opposing end walls, and, for example, prevent portions of end walls of components 76, 78, 80, 82, and 84 from deflecting, for example, under the force of gravity or the force of pressure of fluid 74. As shown, in one aspect, supports 88 may be cylindrical in shape, for example, circular cylindrical, though any cylindrical shape may be used. In one aspect, supports 88 may be hollow as indicated by internal cavities 90 of supports 88. In one aspect, one or more of supports 88 may be hollow. In one aspect, hollow supports 88 may penetrate the end walls of components 76, 78, 80, 82, and 84 or be in fluid communication with through holes in the end walls, for example, where open passages may be provided though components 76, 78, 80, 82, and 84 of device 70 via hollow supports 88.

As also shown in FIGS. 8 through 11, in one aspect, at least one of the components 76, 78, 80, 82, and 84 of device 70 (or of device 10 or of device 40) may include a region of greater thickness or a boss 92, for example, a boss 92 centrally located upon the surface of one or more of the components 76, 78, 80, 82, and 84. One or more bosses 92 may be provided to enhance the structural integrity of device 70 or to introduce variable elasticity to the surface of device 70. The thickness of boss 92 may be from 0.010 to 0.25 inches above the surface of components 76, 78, 80, 82, and 84, but may typically be about 0.125 inches above the surface of components 76, 78, 80, 82, and 84.

In one aspect, as shown most clearly in the bottom view of device 70 shown in FIG. 10, bosses 92 may be omitted. For example, in one aspect, the bottom surface of device 70 (or of device 10 or of device 40), for example, the surface intended to contact the surface being treated, for example, the skin, is devoid of any bosses or extensions in order to enhance contact with the surface being treated. In one aspect, the smooth, substantially uniform contact of surface of device 70 (or of device 10 or of device 40) is believed to enhance the intended treatment of the surface being treated, for example, to enhance heat transfer from the surface and/or to the surface. In addition, in one aspect, the smooth, substantially uniform contact of surface of device 70 (or of device 10 or of device 40) is believed to enhance the comfort or “feel” of the user of device 70 (or of device 10 or of device 40).

In one aspect, as shown in FIGS. 8 through 11, the sidewalls of components 76, 78, 80, 82, and 84 of device 70 may include cavities or recesses 96. Recesses 96 may be provided for aesthetic appearance, to enhance formability or moldability, or to enhance the engagement of device 70 within any enclosure, among other benefits of cavities 96.

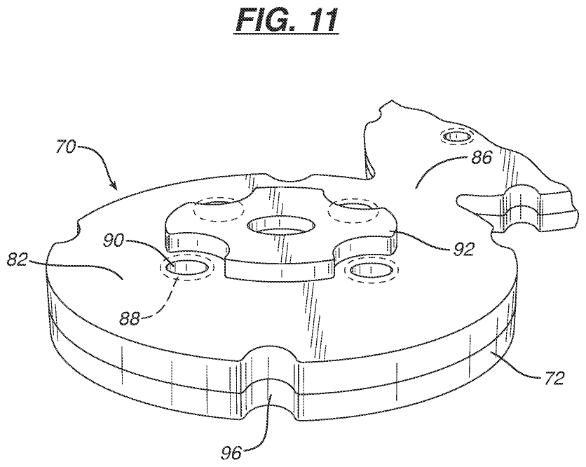

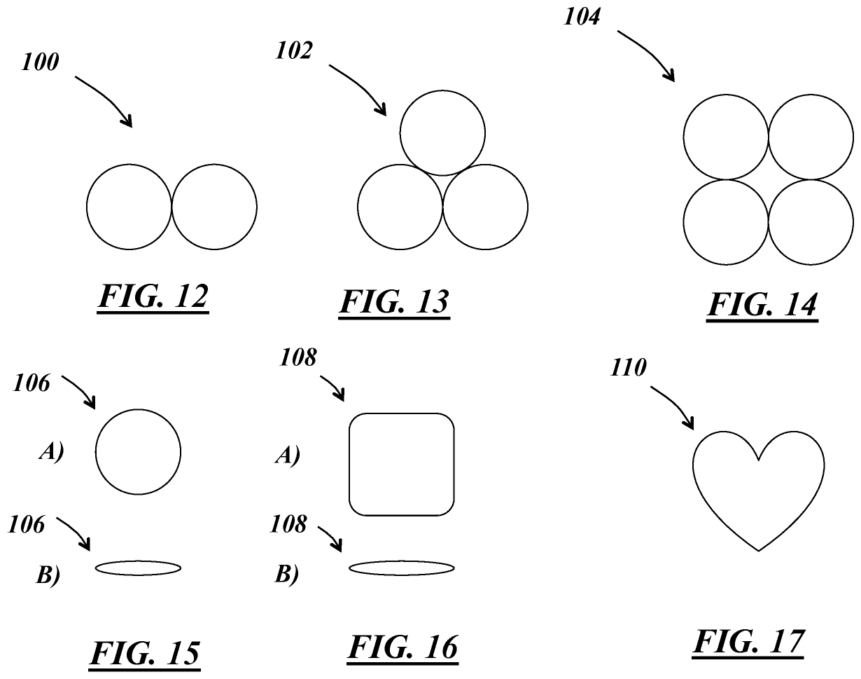

As shown in FIGS. 8 through 11, device 70 may comprise multiple components 76, 78, 80, 82, and 84 arranged in a somewhat uniform radial pattern. However, it is envisioned that, according to aspects of the invention, device 70 (or device 10 or device 40) may take many varying arrangements of components and component shapes as shown schematically in FIGS. 12 through 17.

FIG. 12 is a top plan view of a device 100 that may have one or more of the attributes of devices 10, 40, and 70 according to another aspect of the invention. As shown, device 100 may comprise two or more of devices 10 or devices 40, or two or more of the components 76, 78, 80, 82, and 84 of device 70 shown in FIGS. 8 through 11.

FIG. 13 is a top plan view of a device 102 that may have one or more of the attributes of devices 10, 40, and 70 according to another aspect of the invention. As shown, device 102 may comprise three of the circular cylindrical components 76, 78, 80, 82, and 84 of device 70 shown in FIGS. 8 through 11.

FIG. 14 is a top plan view of a device 104 that may have one or more of the attributes of devices 10, 40, and 70 according to another aspect of the invention. As shown, device 104 may comprise four of the circular cylindrical components 76, 78, 80, 82, and 84 of device 70 shown in FIGS. 8 through 11.

FIG. 15A is a top plan view of a device 106 that may have one or more of the attributes of devices 10, 40, and 70 according to another aspect of the invention. FIG. 15B is a side elevation view of device 106 shown in FIG. 15A. As shown, device 106 may comprise a circular shape having substantially little or no sidewall, and forming a substantially elliptical cross section. For example, device 106 may be formed by fusing or crimping the peripheries of the end walls as known in the art.

FIG. 16A is a top plan view of a device 108 that may have one or more of the attributes of devices 10, 40, and 70 according to another aspect of the invention. FIG. 16B is a side elevation view of device 108 shown in FIG. 16A. As shown, device 108 may comprise a rounded polygonal shape having substantially little or no sidewall but forming a substantially elliptical cross section. For example, device 108 may be formed by fusing or crimping the peripheries of the end walls as known in the art.

FIG. 17 is a top plan view of a device 110 that may have one or more of the attributes of devices 10, 40, and 70 according to another aspect of the invention. As shown, device 110 is suggestive of any one of the myriads of shapes of devices that are envisioned for aspects of the present invention, for example, a heart shaped device shown in FIG. 17. Heart shaped device 110, and any other of the shapes that may be provided may have sidewalls, for example, as shown in FIGS. 1 through 6, or may have substantially no sidewall, as shown in FIG. 15B and 16B, for example, having fused peripheries of the end walls. Other shapes of devices that may be provided according to aspects of the invention may be readily conceivable by those of skill in the art.

According to aspects of the invention, the enclosures of the devices disclosed herein, for example, devices 10, 40, 70, 100, 102, 104, 106, 108, or 110, may comprise a liquid silicone rubber, for example, a cured liquid silicone rubber, such as, a platinum-cured LSR. In one aspect, the enclosures of the devices disclosed herein may comprise an LSR marketed under the trademark True Skin®; however, the enclosures of the devices disclosed herein may comprises any cured liquid silicone rubber (LSR). In one aspect, the cured liquid silicone rubber may be a “medical grade” LSR, as known in the art, for example, an LSR having an elasticity, a viscosity, a “feel,” and/or anti-microbial properties, among other things, that are desirable in aspects of the invention.

However, in one aspect, a liquid silicone rubber marketed under the trademark True Skin® may be preferred. In one aspect, the enclosures disclosed herein may be made from CHT's True Skin® 10 having a 10 Shore A hardness (or durometer) as disclosed in CHT “Technical Data Sheet” for True Skin®10 [Rev-1, Aug. 16, 2017], which is included by reference herein. In one aspect, the enclosures disclosed herein may be made from CHT's True Skin® 20 having a 20 Shore A hardness (or durometer) as disclosed in CHT “Technical Data Sheet” for True Skin® 20 [Rev-1, Aug. 16, 2017], which is included by reference herein. In one aspect, the enclosures disclosed herein may be made from CHT's True Skin® 30 having a 30 Shore A hardness (or durometer) as disclosed in CHT “Technical Data Sheet” for True Skin® 30 [Rev-1, Aug. 16, 2017], which is included by reference herein. As known in the art, any one of the enclosures disclosed herein may be fabricated from a liquid silicon rubber produced when combining two or more components, for example, a CHT True Skin liquid silicone rubber “A” and a CHT True Skin liquid silicone rubber “B” to produce the desired liquid silicone rubber when cured.

It is also envisioned that aspects of the invention may be made from other liquid silicone rubbers, for example, an LSR marketed under the trademark SILBIONE® by Elkem Silicones of Lyon, France, for example, one or more of the LSRs identified in Elkem's “Silbione® Liquid Silicone Rubber (LSR) Elastomers” line card dated June 2018, which is included by reference herein. In another aspect, the enclosures of the invention may be made from an LSR marketed by Dow Corning. In another aspect, enclosures of any one of the devices disclosed herein may be made from an LSR marketed under the trademark DRAGON SKIN™ by Smooth-On, Inc., for example, one or more of the LSRs identified in Smooth-On's Technical Bulletin “Dragon Skin™ Series,” having reference number 041619-JR, which is included by reference herein.

In one aspect, the fluid (for example, liquid) encased in the enclosures of the devices disclosed herein, for example, devices 10, 40, 70, 100, 102, 104, 106, 108, or 110, may comprise a polydimethlysiloxane fluid, for example, a polydimethlysiloxane fluid marketed as a QM Diluent by CHT; however, liquid 74 may comprises any polydimethlysiloxane fluid. However, in one aspect, the polydimethlysiloxane fluid marketed under the trademark QM Diluent may be preferred. In one aspect, the fluid encased in the enclosures of the devices disclosed herein may comprise QM Diluent 50 having a viscosity of about 50 centipoise [cps] as disclosed in CHT “Technical Data Sheet” for QM Diluent 50 [Rev-3, Aug. 7, 2017], which is included by reference herein. In one aspect, the fluid encased in the enclosures of the devices disclosed herein may comprise QM Diluent 100 having a viscosity of about 100 cps as disclosed in CHT “Technical Data Sheet” for QM Diluent 100 [Rev-3, Aug. 7, 2017], which is included by reference herein. In one aspect, the fluid encased in the enclosures of the devices disclosed herein may comprise QM Diluent 1000 having a viscosity of about 1000 cps as disclosed in CHT “Technical Data Sheet” for QM Diluent 1000 [Rev-3, Aug. 7, 2017], which is included by reference herein. In one aspect, the fluid encased in the enclosures of the devices disclosed herein may comprise QM Diluent 5000 having a viscosity of about 5000 cps, for which a “Technical Data Sheet” has not been identified. It is envisioned that fluids of higher viscosity, for example, QM Diluents having a viscosity greater than 5000 cps may be used in aspects of the invention.

It is envisioned that fluids of having other viscosities, for example, QM Diluents provided by CHT, may also be used, for example, by appropriately combining at least two fluids of known viscosity, as known in the art. Accordingly, in one aspect, polydimethlysiloxane fluids of different viscosities, such as, a CHT QM Diluents, can be mixed or otherwise combined to provide a fluid having a viscosity of at least 50 cps that can be used as a fluid in an aspect of the invention. Similarly, in one aspect, polydimethlysiloxane fluids of different viscosities, such as, a CHT QM Diluents, can be mixed or otherwise combined to provide a fluid having a viscosity of at least 100 cps that can be used as a fluid in an aspect of the invention. In one aspect, polydimethlysiloxane fluids of different viscosities, such as, a CHT QM Diluents, can be mixed or otherwise combined to provide a fluid having a viscosity of at least 500 cps that can be used as a fluid in an aspect of the invention. In one aspect, polydimethlysiloxane fluids of different viscosities, such as, a CHT QM Diluents, can be mixed or otherwise combined to provide a fluid having a viscosity of at least 1000 cps, or at least 2000 cps, or at least 3000 cps, or at least 4000 cps, or at least 5000 cps, or at least 6000 cps, or at least 8000 cps that can be used as a fluid in an aspect of the invention.

It is envisioned that a broad range of fluids, including liquids or gases, may be used with aspects of the invention, for example, a liquid or gas compatible with the materials of the enclosures disclosed herein, for instance, fluids that are impermeable to the material of the enclosure disclosed herein or fluids having a boiling point where the fluid will not evaporate under the expected working conditions. In one aspect, the liquid may comprise a broad range of viscosities, for example, from less than 50 cps to greater than 5000 cps. In other aspects, the liquid may be water, for example, distilled water.

Though not shown in FIGS. 1 through 17, it is envisioned that any one of more of the devices disclosed herein may be enhanced with appropriate text and/or logos. For example, instructions for use and/or marketing information or logos may be applied to a surface of the devices disclosed herein. In addition, it is envisioned that the aesthetic appeal of aspects of the invention may be enhanced by introducing dyes or colorants to the enclosures and/or to the fluid encased within the enclosures.

In one aspect of the invention, the devices disclosed herein may be enhanced with an appropriate mountings or attachment mechanisms or devices. For example, in one aspect, any one of the devices disclosed herein may include a mounting device, for example, one or more straps (with or without connectors), holders, and/or retainers. In another aspect, any one of the devices disclosed herein may be enhanced with an attachment or retention mechanism or device, for example, one or more fasteners. In one aspect, any one of the devices disclosed herein may include one or more hook and loop fasteners, for example, a Velcro-type hook and loop fastener, as known in the art. The hook and loop fastener may be used to facilitate or enhance mounting or locating any one of the devices disclosed herein, for example, to a mating hook and look fastener. In one aspect, a hook and loop fastener may be attached to any one of the devices disclosed herein with mounting hardware and/or with an adhesive. In another aspect, a fastener, such as, a hook and loop faster, may be molded into any one of the enclosures of the devices disclosed herein. For example, in one aspect, a hook and loop fastener may be molded into any one or more of the bosses 92 shown in FIGS. 8 through 11 during the molding process.

As disclosed herein surface treatment devices, for example, heating, cooling, and/or comforting devices or packs, are disclosed herein that provide enhanced performance and comfort to the user. Though aspects of the invention have been primarily disclosed for use in treating, for example, heating, cooling, or comfort of the human skin and the underlying tissue and/or organs, aspects of the invention may be applied when effective for treating any medical, non-medical, industrial, commercial, and/or residential surface.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1-20. (canceled)

21. A surface treatment device comprising:
a flexible, fluid impermeable enclosure comprising a synthetic amorphous silica; and
a polydimethlysiloxane fluid contained in the enclosure.

22. The device as recited in claim 21, wherein the device comprises a heating device.

23. The device as recited in claim 22, wherein the heating device comprises a heating pack.

24. The device as recited in claim 21, wherein the device comprises a cooling device.

25. The device as recited in claim 24, wherein the cooling device comprises a cooling pack.

26. The device as recited in claim 21, wherein the synthetic amorphous silica comprises CHT True Skin 10 liquid silicone rubber.

27. The device as recited in claim 21, wherein the synthetic amorphous silica comprises CHT True Skin liquid silicone rubber produced by combining at least two liquids.

28. The device as recited in claim 27, wherein the at least two liquids comprise a CHT True Skin liquid silicone rubber A and a CHT True Skin liquid silicone rubber B.

29. The device as recited in claim 21, wherein the polydimethlysiloxane fluid comprises a low viscosity CHT QM Diluent.

30. The device as recited in claim 29, wherein the CHT QM Diluent comprises a CHT QM Diluent having a viscosity of at least 50 cps.

31. The device as recited in claim 29, wherein the CHT QM Diluent comprises a CHT QM Diluent having a viscosity of at least 100 cps.

32. The device as recited in claim 29, wherein the CHT QM Diluent comprises a CHT QM Diluent having a viscosity of at least 500 cps.

33. The device as recited in claim 29, wherein the CHT QM Diluent comprises a CHT QM Diluent having a viscosity of at least 1,000 cps.

34. The device as recited in claim 29, wherein the CHT QM Diluent comprises a CHT QM Diluent having a viscosity of at 2,000 cps.

35. The device as recited in claim 29, wherein the CHT QM Diluent comprises a CHT QM Diluent having a viscosity of at least 3,000 cps.

36. The device as recited in claim 29, wherein the CHT QM Diluent comprises a CHT QM Diluent having a viscosity of at least 4,000 cps.

37-40. (canceled)

41. A method for treating a surface comprising:
heating or cooling a flexible, fluid impermeable enclosure comprising a synthetic amorphous silica and containing a polydimethlysiloxane fluid;
placing the heated or cooled flexible enclosure upon the surface; and
conducting thermal energy from the enclosure to the surface or conducting thermal energy from the surface to the enclosure.

42. The method as recited in claim 41, wherein the treating comprises heating the surface, and wherein the method comprises conducting thermal energy from the enclosure to the surface.

43. The method as recited in claim 41, wherein the treating comprises cooling the surface, and wherein the method comprises conducting thermal energy from the surface to the enclosure.

44. The method as recited in claim 41, wherein heating comprises heating in a heating device and cooling comprises cooling in a cooling device.

45-63. (canceled)
